(19) United States
(12) Patent Application Publication
(10) Pub. No.: US 2023/0211125 A1
(43) Pub. Date: Jul. 6, 2023

(54) EXPANDABLE SHEATH INCLUDING REVERSE BAYONET LOCKING HUB

(71) Applicant: EDWARDS LIFESCIENCES CORPORATION, Irvine, CA (US)

(72) Inventors: Nasser William Saleh, Stockton, CA (US); Ilan Tamir, Irvine, CA (US); Salvatore Manzella, JR., Aliso Viejo, CA (US); Ofir Witzman, Harish (IL); Oren Cohen, Kadima (IL)

(21) Appl. No.: 18/120,496

(22) Filed: Mar. 13, 2023

Related U.S. Application Data
(63) Continuation of application No. PCT/US2021/050006, filed on Sep. 13, 2021.
(60) Provisional application No. 63/077,899, filed on Sep. 14, 2020.

(51) Int. Cl. A61M 25/00; A61F 2/24

(52) U.S. Cl. CPC A61M 25/0097; A61F 2/2427; A61M 25/06

(57) ABSTRACT

A sheath locking system comprising an introducer locking hub comprising a hub body defining a locking channel for engaging a corresponding guide on a sheath locking sleeve. The sheath locking sleeve removably coupled to the introducer locking hub via engagement between the guide and the locking channel. The sheath locking sleeve movable between an unlocked position where the sheath locking sleeve is rotationally and axially movable with respect to the introducer locking hub, and a locked position where the sheath locking sleeve is axially fixed with respect to the introducer locking hub.

### CROSS-REFERENCE TO RELATED APPLICATIONS

This application is a continuation of International Application No. PCT/US2021/050006, filed Sep. 13, 2021, which claims the benefit of U.S. Provisional Application No. 63/077,899, filed Sep. 14, 2020, the content of which are incorporated herein by reference in their entirety.

### FIELD

The present application is directed to a sheath for use with catheter-based technologies for repairing and/or replacing heart valves, as well as for delivering an implant, such as a prosthetic valve to a heart via the patient's vasculature.

### BACKGROUND OF THE INVENTION

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques. Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device. Expandable introducer sheaths are disclosed in U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. application Ser. No. 14/880,109, entitled “Expandable Sheath,” U.S. application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. application Ser. No. 15/997,587, entitled “Expandable Sheath for Introducing an Endovascular Delivery Device into a Body,” U.S. application Ser. No. 16/378,417, entitled “Expandable Sheath,” the disclosures of which are herein incorporated by reference.

Conventional methods of accessing a vessel, such as a femoral artery, prior to introducing the delivery system include dilating the vessel using multiple dilators or sheaths that progressively increase in diameter. Typically, the introducer is inserted into the sheath during preparation and both are then inserted into the vessel. Due to the need for a smooth transition from the introducer to the sheath, it is vital that the change in diameter of the introducer occurs distal to the tip of the sheath, such that the tip of the sheath fits snugly around the diameter.

During insertion of the sheath and introducer, it is possible that the introducer can move backwards within the sheath, eliminating the aforementioned snug fit and creating a lip between the sheath tip and the smaller outer diameter of the introducer. This lip/gap can lead to severe vessel trauma during insertion.

Moreover, some procedures, such as a transseptal approach for mitral valve replacement/repair, require prolonged dilation of incisions in heart tissue and a curving/bending of the sheath to access the treatment site, prolonging procedure time and recovering and increasing risk of trauma to vessels and heart tissue.

Accordingly, there remains a need for further improvements in expandable introducer sheath for endovascular systems used to implant valves and other prosthetic devices.

### SUMMARY OF THE INVENTION

The sheath locking system disclosed herein comprises a an introducer locking hub comprising a hub body having a proximal end and a distal end and defining a central lumen extending longitudinally between the proximal end and the distal end, and a locking channel disposed on the hub body; and a sheath locking sleeve removably coupled to the introducer locking hub, the sheath locking sleeve comprising a sleeve body having a proximal end and a distal end and defining a central lumen extending longitudinally between the proximal end and the distal end, a guide disposed on an outer surface of the sleeve body, wherein the guide is movable within the locking channel between an unlocked position where the sheath locking sleeve is rotationally and axially movable with respect to the introducer locking hub, and a locked position where the sheath locking sleeve is axially fixed with respect to the introducer locking hub.

A method of delivering a prosthetic device to a procedure site is disclosed herein, the method includes: providing an introducer locking hub having an elongated introducer coupled to a hub body of the locking hub, the introducer locking hub including a locking channel disposed in the hub body; advancing a sheath locking sleeve to a position adjacent a distal end of the introducer locking hub such that a guide projecting from an outer surface of the sheath locking sleeve is received within a locking channel opening on the introducer locking hub, the sheath locking sleeve coupled to an expandable delivery sheath, where advancing the sheath locking sleeve to a position adjacent the distal end of the introducer locking hub includes advancing the introducer axially within the central lumen of the expandable delivery sheath; rotating the introducer locking hub in a first direction with respect to the locking sleeve to move the guide along the locking channel into a locked position; inserting the coupled sheath and introducer at least partially into the vasculature of the patient; rotating the introducer locking hub in a second direction with respect to the locking sleeve to slide the guide along the locking channel into an unlocked position; disengaging the introducer locking hub from the locking sleeve; withdrawing the introducer from the central lumen of the sheath; advancing a medical device through the central lumen of the sheath; and delivering the medical device to a procedure site via the central lumen of the sheath.

A method of securing a delivery sheath to an introducer in a device for prosthetic heart valve delivery device, the method comprising: providing an introducer locking hub having an elongated introducer coupled to a hub body of the locking hub, the introducer locking hub including a locking channel disposed in the hub body; advancing a sheath locking sleeve to a position adjacent a distal end of the introducer locking hub such that a guide projecting from an outer surface of the sheath locking sleeve is received within a locking channel opening on the introducer locking hub, the sheath locking sleeve coupled to an expandable delivery sheath, where advancing the sheath locking sleeve to a position adjacent the distal end of the introducer locking hub includes advancing the introducer axially within the central lumen of the expandable delivery sheath; rotating the introducer locking hub in a first direction with respect to the locking sleeve to move the guide along the locking channel into a locked position; rotating the introducer locking hub in a second direction with respect to the locking sleeve to move the guide along the locking channel into an unlocked position.

An expandable introducer sheath for deploying a medical device is disclosed herein. The sheath comprising a first layer including a central lumen extending axially therethrough; a resilient elastic layer radially outward of the first layer, the elastic layer being configured to apply radial force to the first layer; and wherein when a medical device is passed through the sheath, the diameter of the sheath expands from an initial diameter to an expanded diameter around the medical device, wherein the sheath resiliently returns to the initial diameter by radial force applied by the elastic layer upon passage of the medical device, wherein a distal tip of the sheath is configured to bend is a direction away from a longitudinal axis of the sheath.

A method for controlling articulation/bending of a delivery sheath is disclosed herein.

The method comprising: providing an expandable introducer sheath with a central lumen extending therethrough, where a distal tip portion of the sheath is more flexible than a proximal portion of the sheath; applying a force to a pull wire coupled to a distal end of the sheath to bend the distal tip portion in a direction away from a longitudinal axis of the sheath; releasing the force on the pull wire to return the distal tip portion to back towards the longitudinal axis of the sheath.

A method for controlling articulation/bending of a delivery sheath is disclosed herein. The method comprises: providing an expandable introducer sheath with a central lumen extending therethrough, where a distal tip portion of the sheath is more flexible than a proximal portion of the sheath; inserting a stylet into the central lumen of the sheath, where the stylet includes a curved portion for effecting a curve on the sheath; aligning the curved portion of the stylet with the distal tip portion to curve the distal tip portion in a direction away from the longitudinal axis of the sheath; removing the stylet at least partially from the central lumen of the sheath such that the curved portion of the stylet is no longer aligned with the distal tip portion of the sheath, to return the distal tip portion back towards the longitudinal axis of the sheath.

A method of delivering a medical device is disclosed herein. The method comprises: inserting the sheath at least partially into the blood vessel of the patient; advancing a distal end of the sheath to a first location proximate the treatment site; curving the distal end of the sheath; advancing the distal end of the sheath to the treatment site; advancing a medical device through the central lumen of the sheath to the treatment site; locally expanding the sheath from an initial condition/diameter to a locally expanded condition/diameter by the outwardly directed radial force of the medical device; locally contracting the sheath from the locally expanded condition at least partially back to the initial condition using inwardly directed radial force an elastic feature of the sheath; and delivering the medical device to the treatment site.

Some aspects further include advancing the distal end of the sheath through the opening patient's heart tissue.

### BRIEF DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross sectional view of the sheath hub, introducer locking hub, and sheath locking sleeve of FIG. 2.

FIG. 5B is a cross sectional view of the introducer cap, the sheath hub, the introducer locking hub, the sheath locking sleeve of FIG. 2.

FIG. 6 is a cross sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIG. 3.

FIG. 12 is a magnified view of a portion of the expandable sheath of FIG. 3.

FIG. 13A is a magnified view of a portion of the expandable sheath of FIG. 3 with the outer layer removed for purposes of illustration.

FIG. 13B is a magnified view of a portion of the braided layer of the sheath of FIG. 3.

FIG. 14 is a magnified view of a portion of the expandable sheath of FIG. 3 illustrating expansion of the sheath as a prosthetic device is advanced through the sheath.

FIGS. 15A-15C are side views of the expandable sheath of FIG. 3 including a delivery device and implant.

### DETAILED DESCRIPTION OF THE INVENTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed examples of an expandable sheath can minimize trauma to the vessel by allowing for temporary expansion of a portion of the introducer sheath to accommodate the delivery system, followed by a return to the original diameter once the device passes through. Disclosed examples of the introducer sheath prevent the introducer from separating from the sheath during insertion by locking of the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present examples can reduce the length of time a procedure takes, as well as reduce the risk of a longitudinal or radial vessel tear, or plaque dislodgement because only one sheath is required, rather than several different sizes of sheaths. Examples of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

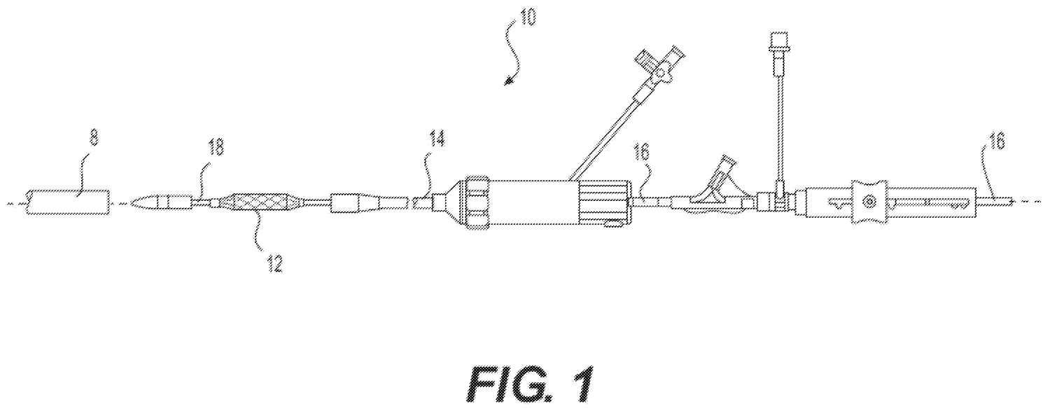

FIG. 1 illustrates an exemplary sheath 8 in use with a representative delivery apparatus 10, for delivering an implant 12, or other type of implantable, to a patient. The delivery apparatus 10 can include a steerable guide catheter 14 (also referred to as a flex catheter) and a balloon catheter 16 extending through the guide catheter 14. The guide catheter 14 and the balloon catheter 16 in the illustrated aspect are adapted to slide longitudinally relative to each other to facilitate delivery and positioning of the implant 12 at an implantation site in a patient's body, as described in detail below. The sheath 8 is an elongate, expandable tube that can include a hemostasis valve at the proximal end of the sheath to stop blood leakage.

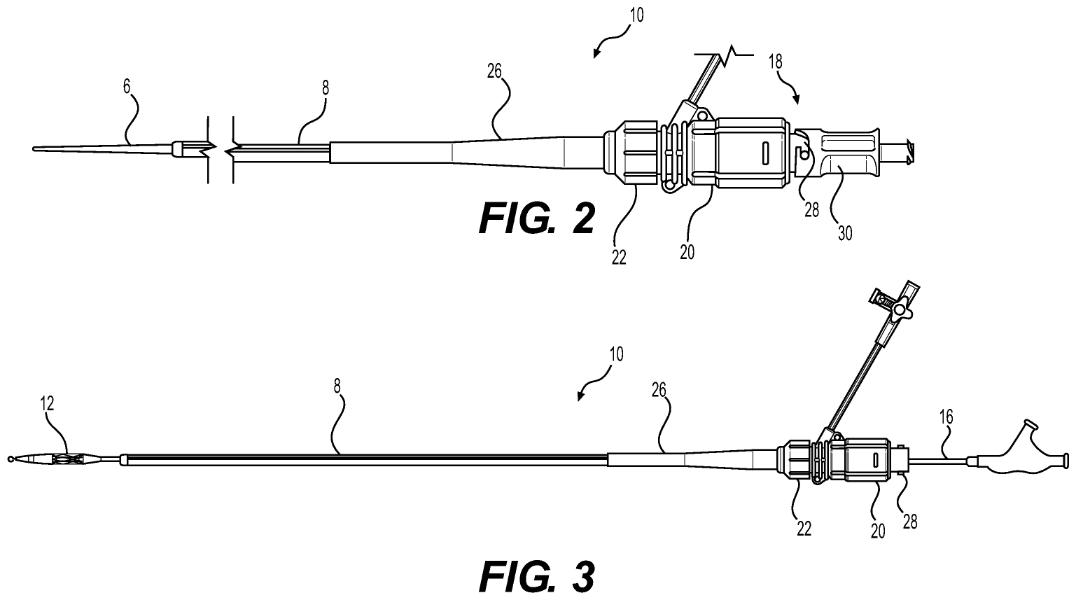

FIG. 2 illustrates the sheath 8 of FIG. 1 including a sheath locking system 18 which prevents axial and rotational translation of the introducer 6 with respect to the sheath 8. The sheath locking system 18 keeps the introducer 6 fixed with respect to the sheath 8 during insertion without requiring a physician or technician to hold the introducer 6 and the sheath 8 in place at the distal end. The sheath locking system 18 includes a sheath locking sleeve 28 coupled to the sheath 8 via sheath hub 20 and an introducer locking hub 30 and introducer 6. The sheath locking sleeve 28 engages the introducer locking hub 30 and is moveable between a locked and unlocked position, thereby fixing the position of the introducer 6 and the sheath 8 and preventing movement therebetween during insertion. As will be described in more detail below, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupling the introducer locking hub 30 to the sheath hub 20. As will be described in more detail below, the sheath locking sleeve 28 includes a guide 31 that engages a locking channel 38 provided on the introducer locking hub 30. The guide 31 moves within the locking channel 38 between an unlocked position, where the sheath locking sleeve 28 is rotationally and axially movable with respect to the introducer locking hub 30, and a locked position (FIG. 2), where the sheath locking sleeve 28 is axially fixed with respect to the introducer locking hub 30.

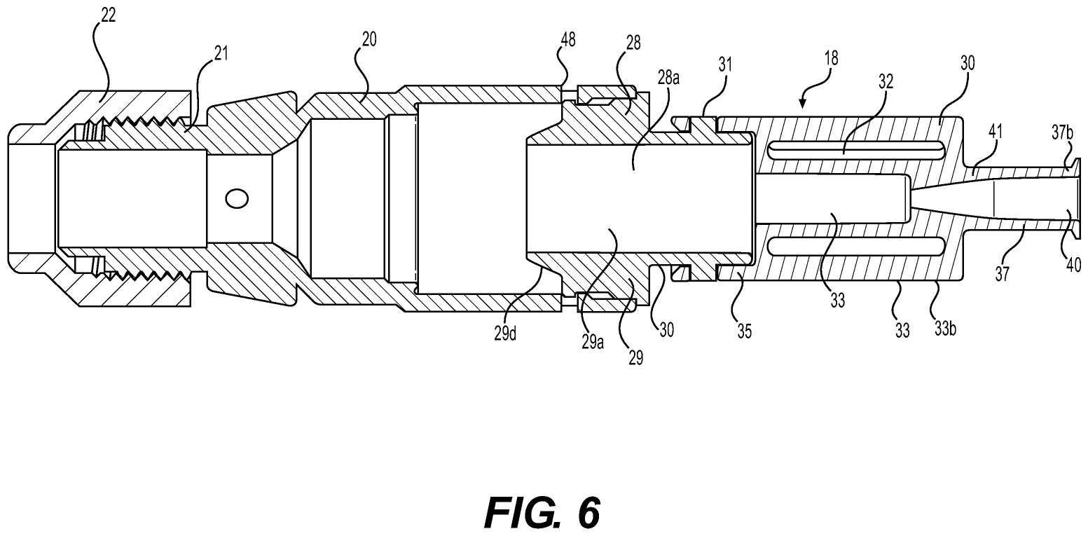

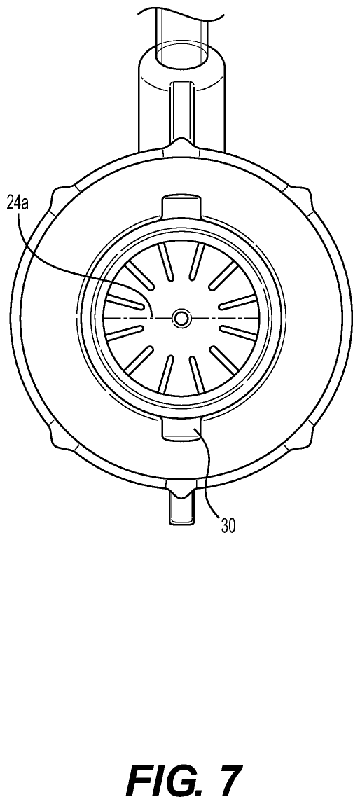

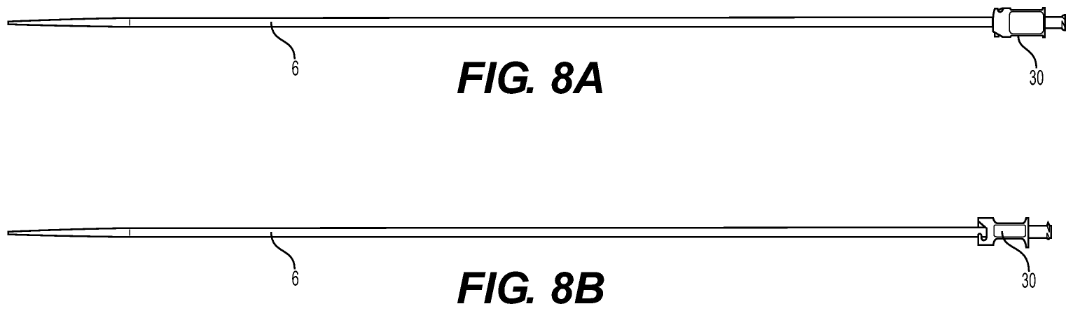

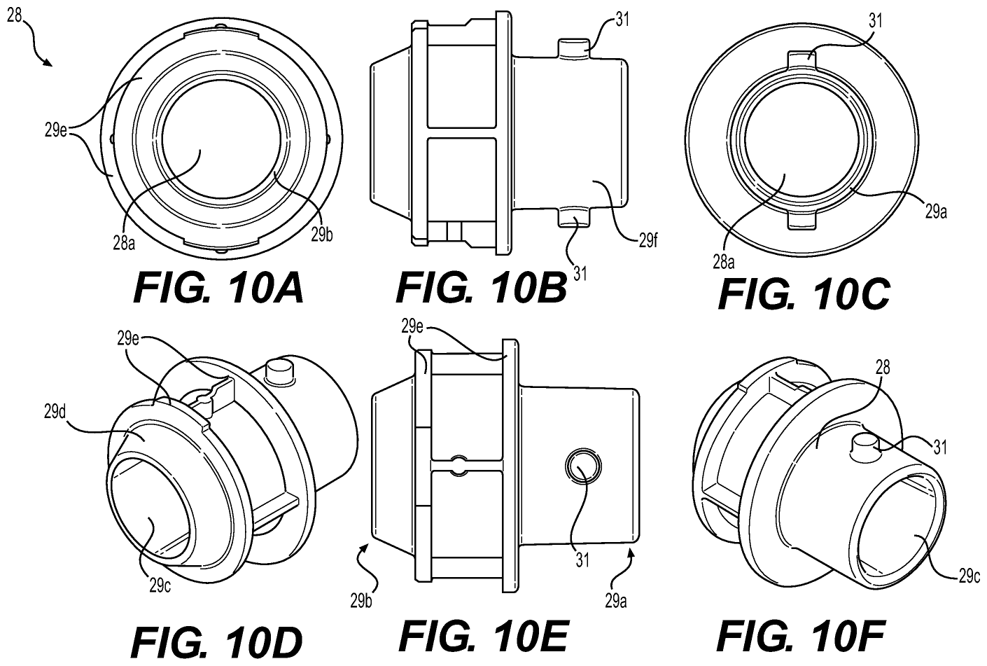

The sheath locking sleeve 28 is illustrated, for example, in FIGS. 10A-10F. The sheath locking sleeve 28 includes an elongated sleeve body 29 with a central lumen 28a extending longitudinally between the proximal end 29a and distal end 29b of the sleeve body 29. As provided in FIG. 6, the central lumen 28a defines a generally cylindrical inner surface 29c of the sheath locking sleeve 28. The central lumen 28a has a diameter of at least 0.3″. In some examples, the diameter ranges between 0.3″ and 0.6″. Preferably, the diameter is about 0.40″. The distal end 29b of the sleeve body 29 also has a frustoconical outer surface 29d that tapers about the distal end 29b to help with positioning the sheath locking sleeve 28 within the sheath hub 20 and abutting the seal assembly 24 (FIGS. 5B and 5B). The sheath locking sleeve 28 also has a plurality of interface diameters 29e that extend radially from the outer surface 29d of the sleeve body 29 around (all or a portion of) the circumference of the sheath locking sleeve 28. As illustrated in FIG. 5A, these interface diameters 29e are sized and configured to engage corresponding recesses and/or slots 48 provided in the sheath hub 20 for securing the sheath locking sleeve 28 to the sheath hub 20.

The sheath locking sleeve 28 includes a guide 31 projecting from the outer surface 29f of the sheath locking sleeve 28. The guide 31 engages a corresponding shaped locking channel 38 in the introducer locking hub 30. The guide 31 extends radially from the outer surface 29f and at least partially around the circumference of the outer surface 29f. As provided in FIG. 6, the top surface of the guide 31 is does not extend beyond the outer surface of the introducer locking hub 30 when the sheath locking sleeve 28 and the introducer locking hub 30 are coupled. For example, the height of the guide 31 corresponds to the wall thickness of the introducer locking hub 30 proximate the guide when the sheath locking sleeve 28 and the introducer locking hub 30 are coupled. In another example, the top surface of the guide 31 is recessed with respect to the outer surface of the introducer locking hub 30. That is, the height of the guide is less than the wall thickness of the introducer locking hub 30. In other examples, the height of the guide 31 is greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050″ and about 0.10.″ In some examples that height/axial length of the guide 31 is about 0.075″.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05″ to about 0.20″. Preferably the guide 31 has a diameter/width of about 0.100″.

In general, the sheath locking sleeve 28 can be formed from polycarbonate, but in other examples the sheath locking sleeve 28 can be formed from rigid plastic, or any other material suitable for providing a strong locking connector for an introducer 6 (metal, composite, etc.)

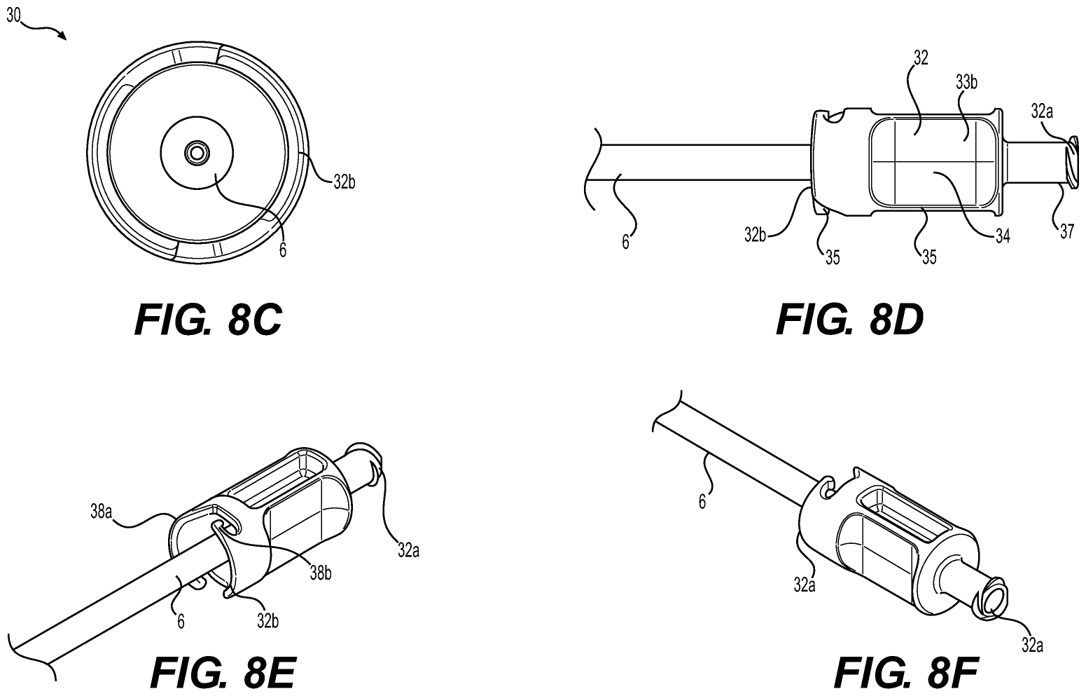

FIGS. 8A-8F illustrate the introducer locking hub 30 with the introducer 6 coupled thereto. Example introducer sheaths are described, for example in U.S. Pat. Nos. 8,690,936 and 8,790,387, the disclosures of which are incorporated herein by reference. As provided in the cross-section views of FIGS. 5A and 5B, the introducer 6 is coupled to the introducer locking hub 30 and extends beyond the distal end of the introducer locking hub 30 body. When coupled to the sheath hub 20, the introducer 6 extends through the central lumen 28a of the sheath locking sleeve 28, the sheath hub 20 and the central lumen of the sheath 8. As will be descried below, the sheath 8 generally comprises a radially expandable tubular structure. Passage of the introducer 6 through the sheath 8 and into a patient's vessel causes the vessel to radially expand to about the diameter of the sheath 8. That is, the diameter of the central lumen of the sheath 8 is generally about the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. The introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to or less than the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (e.g., an adhesive), a weld, a thermal process, and/or any other suitable coupling process known in the art.

As described above, the introducer 6 has a central lumen that aligns with the central lumen 45 of the introducer locking hub 30. This joined lumen allows for the passage of surgical equipment and/or medical devices to the treatment site. In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In other examples, the diameter of the central lumen 45 at the distal end of the introducer locking hub 30 is slightly larger than the diameter of the central lumen passing through the introducer 6. The central lumen 45 can also a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device through the introducer locking hub 30 and into the central lumen of the introducer 6.

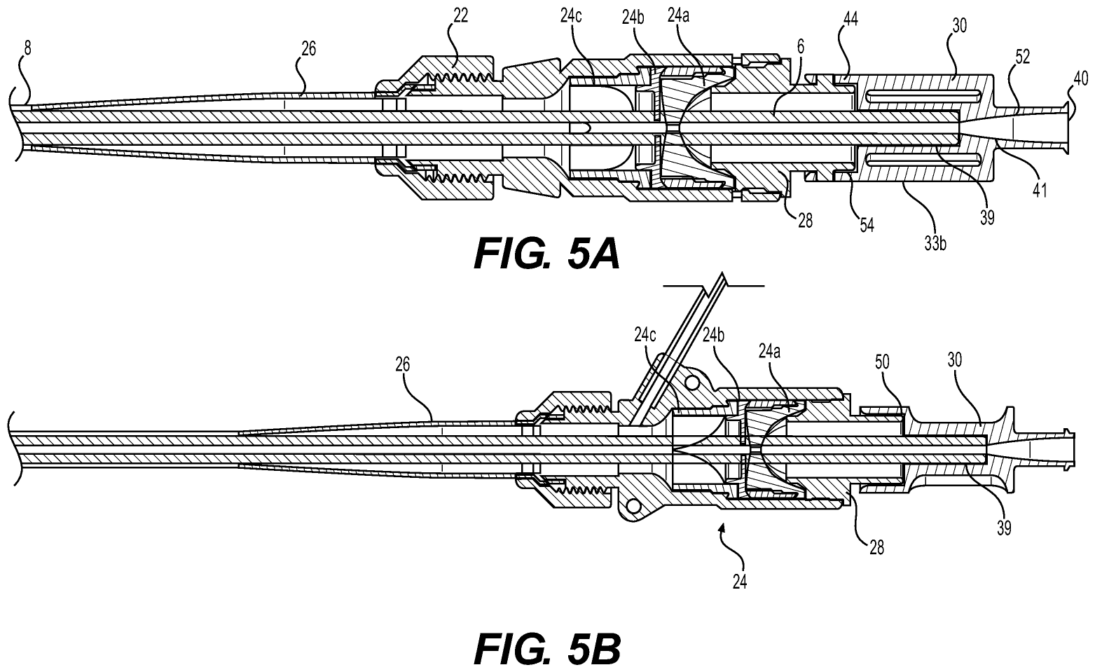

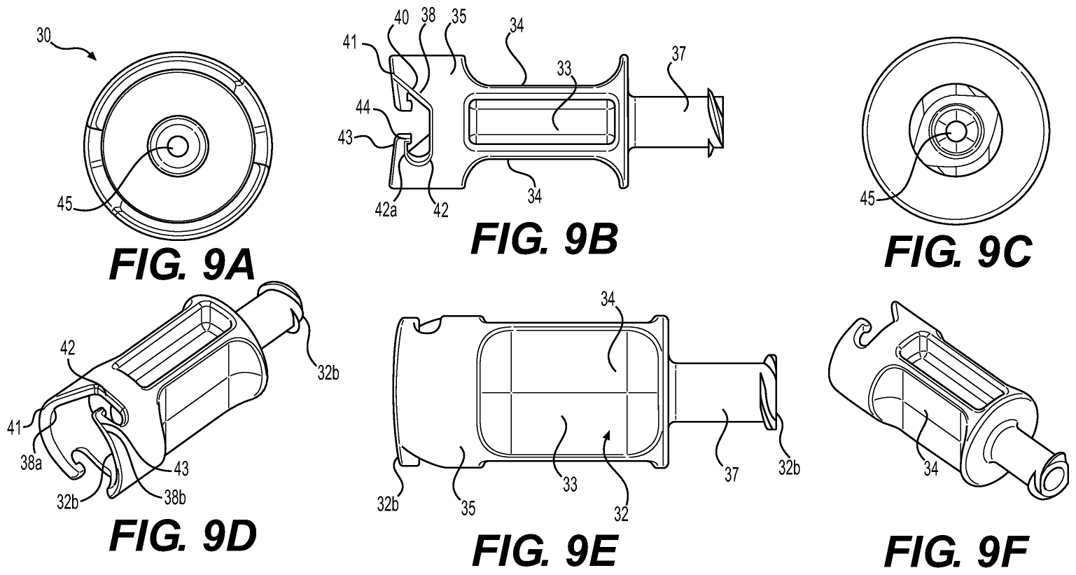

FIGS. 2-6 illustrate the introducer locking hub 30 coupled to the sheath locking sleeve 28. FIGS. 8A-8F provide the introducer locking hub 30 coupled to the introducer 6. FIGS. 9A-9F provide multiple view of the introducer locking hub 30. As described above, the introducer 6 is fixedly coupled to the introducer locking hub 30, and the introducer locking hub 30 couples with the sheath locking sleeve 28 to fix the position the introducer 6 (axially and rotationally) with respect to the sheath locking sleeve 28/sheath 8.

The introducer locking hub 30 includes a hub body 32 having a proximal end 32a and a distal end 32b and defining a central lumen 45 extending therethrough. The introducer locking hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 33b. In some examples, the recessed opening 39 has a diameter ranging between 0.15″ and about 0.25″. In some examples, the recessed opening 39 has a diameter ranging between 0.17″ and about 0.20″. In some examples, the recessed opening has a diameter of about 0.194″.

The third (proximal) portion 37 of the introducer locking hub 30 includes the decreasing tapered portion 41 of the central lumen 45. The decreasing taper portion 41 defining a frustoconical shape with decreasing taper/diameter from the proximal to the distal end of the sheath. It is contemplated that the tapered portion 41 has a minimum diameter of about 0.007″ and a maximum diameter of about 0.194″.

As illustrated in FIG. 5A-5B, when coupled the central lumen 28a of the sheath locking sleeve 28 is aligned with the central lumen 45 of the introducer locking hub 30. In some examples, the central lumen 28a of the sheath locking sleeve 28 is coaxial with the central lumen 45 of the introducer locking hub 30. When coupled, the proximal end of the sheath locking sleeve 28 is received within the central lumen 45 of the introducer locking hub 30. The proximal end surface of the sheath locking sleeve 28 is adjacent a shoulder 50 provided on an inner surface of the central lumen 45 of the introducer locking hub 30. As illustrated in FIGS. 5A and 5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be consider either a component of the first portion 52 of the central lumen 45, or a separate component of the central lumen 45 locate between the first (proximal) portion 52 and the second (distal) portion 54. When the sheath locking sleeve 28 and introducer locking hub 30 are coupled, at least a portion of the sleeve body 29 of the sheath locking sleeve 28 is received within the second portion 54 (larger portion) of the central lumen 45 of the introducer locking hub 30. The central lumen 28a of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described generally above, the sheath locking sleeve 28 couples to the introducer locking hub 30 via engagement between the guide 31 on the sheath locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the sheath locking sleeve 28 with the introducer locking hub 30. Illustrated in FIG. 9B, the locking channels 38 provide an interface to secure the sheath locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the sheath locking sleeve 28 in an axial and circumferential direction along the side wall of the guide portion 40 towards the locking portion 42 upon rotation of the introducer locking hub 30 and/or the sheath locking sleeve 28. The locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than a length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 42a of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal surface of the distal end 32b of the introducer locking hub 30 can include features for biasing the guide 31 towards the locking channel 38. For example, the distal end of the introducer locking hub 30 can include a tapered surface angled toward an opening of the locking channel 38. As illustrated in FIG. 9B, the distal end 32b of the introducer locking hub 30 includes a first tapered surface (decreasing tapered portion 41) angled towards a leading edge of the opening of the locking channel 38 and a second tapered surface 43 angled towards the trailing edge of the opening of the locking channel 38.

In use, engagement between the guide 31 and the guide portion 40 of the locking channel 38 is configured to bias the sheath locking sleeve 28 in a proximal axial direction toward the proximal end of the introducer locking hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the sheath locking sleeve 28 in a distal axial direction toward the distal end of the introducer locking hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface 33b of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the introducer locking hub body 32, i.e., a shape having a smaller diameter/width center portion and larger diameter/width end portions. In an example system, the gripping surfaces 34 are provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate, but in other examples the introducer locking hub 30 can be formed from rigid plastic, or any other material suitable for providing a locking mechanism for an introducer 6 (metal, composite, etc.).

FIGS. 2-6 illustrate an example sheath hub 20. As described above, the sheath 8 is coupled to the sheath hub 20 which in turn is removably coupled to the sheath locking sleeve 28. The sheath hub 20 provides a housing for necessary seal assemblies and an access point for a secondary lumen (e.g., fluid lumen) in fluid communication with the central lumen of the sheath hub 20.

The sheath hub 20 further has receiving slots 48. The receiving slots 48 are openings which extend around a portion of the diameter of the sheath hub 20 and are sized and configured to accept the interface diameters 29e. Coupling between the receiving slots 48 and the interface diameters 29e of the sheath locking sleeve 28 axially and rotationally fixes the sheath locking sleeve 28 and the sheath hub 20 relative to each other.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

The seal assembly 24 as described above and as shown in FIGS. 5A and 5B is included in sheath hub 20. The seal assembly 24 includes proximal seal 24a, intermediate seal 24b, and distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

As illustrated in FIG. 2, the sheath 8 includes a seal tube 26/strain relief portion. The seal tube 26 is coupled to the distal end of the sheath hub 20 and creates a smooth transition surface between the sheath 8 and the sheath hub 20. The frustoconical seal tube 26 body has a proximal end and a distal end and a central lumen extending longitudinally therethrough. The seal tube 26 tapers from the proximal end to the distal end such that the diameter of the seal tube 26 at the proximal end is greater than the diameter of the seal tube 26 at the distal end of the seal tube 26.

A method for delivering a prosthetic device to a procedure site such that axial movement between the introducer 6 and the sheath 8 is eliminated is described below. Preventing gapping between the introducer 6 and the sheath 8 during insertion reduces the risk of trauma to the patient's vasculature. FIG. 2 shows the example device for delivering the prosthetic device.

The method includes providing an introducer locking hub 30 having an elongated introducer 6 coupled to the introducer locking hub body 32 of the introducer locking hub 30. As described above the introducer locking hub 30 including a locking channel 38 disposed in the introducer locking hub body 32. The sheath locking sleeve 28 is advanced to a position adjacent a distal end of the introducer locking hub 30 such that a guide 31 projecting from an outer surface of the sheath locking sleeve 28 is received within the opening to the locking channel 38. Advancing the sheath locking sleeve 28 to a position adjacent the distal end of the introducer locking hub 30 also includes advancing the introducer 6 axially within the central lumen of the expandable delivery sheath 8.

The introducer locking hub 30 is then rotated in a first direction with respect to the sheath locking sleeve 28 to move the guide 31 along the locking channel 38 into a locked position. In particular, moving the guide 31 into the locked position includes rotating the introducer locking hub 30 to move the guide 31 along a guide portion 40 of the locking channel 38 toward a locking portion 42. Further rotation of the introducer locking hub 30 directs the guide 31 into the locking portion 42 of the locking channel 38, the locking portion 42 configured to securely engage the guide 31 and fix the axial position of the introducer locking hub 30 with respect to the sheath locking sleeve 28. Where the locking channel 38 includes a catch 44, rotation of the introducer locking hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

The coupled sheath 8 and introducer 6 are then inserted, at least partially, into the vasculature of the patient.

Once positioned, the introducer locking hub 30 is rotated is a second, opposite, direction with respect to the sheath locking sleeve 28. Rotating the introducer locking hub 30 in the second direction causes the guide 31 to slide along the locking channel 38, from the locking portion 42 toward the guide portion 40. In particular, rotating of the introducer locking hub 30 in the second direction directs the guide 31 out of the locking portion 42 of the locking channel 38 and through the guide portion 40 and releases the introducer locking hub 30 from the sheath locking sleeve 28. Where the locking channel 38 includes a catch 44, rotation of the introducer locking hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position.

The introducer locking hub 30 is then disengaged from the sheath locking sleeve 28 and the introducer 6 is withdrawn from the central lumen of the sheath 8. With the central lumen of the sheath 8 clear, the medical device (e.g., implant 12) is advanced through the central lumen of the sheath 8. The medical device (implant 12) is delivered to the procedure site via the central lumen of the sheath 8.

A method of securing a delivery sheath to an introducer in a device for prosthetic heart valve delivery device is disclosed herein. The method comprises providing an introducer locking hub 30 having an elongated introducer 6 coupled thereto and including a locking channel 38 disposed in the introducer locking hub body 32. The sheath locking sleeve 28 is advanced to a position adjacent a distal end of the introducer locking hub 30 such that a guide 31 projecting from an outer surface of the sheath locking sleeve 28 is received within an opening of the locking channel 38. Advancing the sheath locking sleeve 28 to a position adjacent the distal end of the introducer locking hub 30 also includes advancing the introducer 6 axially within the central lumen of the expandable delivery sheath 8.

The introducer locking hub 30 is then rotated in a first direction with respect to the sheath locking sleeve 28 to move the guide 31 along the locking channel 38 into the locked position. In particular, moving the guide 31 into the locked position includes rotating the introducer locking hub 30 to move the guide 31 along a guide portion 40 of the locking channel 38 toward a locking portion 42. Further rotation of the introducer locking hub 30 directs the guide 31 into the locking portion 42 of the locking channel 38, the locking portion 42 configured to securely engage the guide 31 and fix the axial position of the introducer locking hub 30 with respect to the sheath locking sleeve 28. Where the locking channel 38 includes a catch 44, rotation of the introducer locking hub 30 in the first direction causes the guide 31 to overcome the bias force of the catch 44 and advance the guide 31 beyond the catch 44 into the locking portion 42, where the catch 44 secures the guide 31 within the locking portion 42 thereby fixing the axial location of the sheath 8 with respect to the introducer 6.

To unlock the introducer locking hub 30 from the sheath locking sleeve 28, the introducer locking hub 30 is rotated in a second, opposite, direction with respect to the sheath locking sleeve 28. Rotating the introducer locking hub 30 in the second direction causes the guide 31 to side along the locking channel 38, from the locking portion 42 toward the guide portion 40. In particular, rotating of the introducer locking hub 30 in the second direction directs the guide 31 out of the locking portion 42 of the locking channel 38 and through the guide portion 40 to release the introducer locking hub 30 from the sheath locking sleeve 28. Where the locking channel 38 includes a catch 44, rotation of the introducer locking hub 30 in the second direction causes the guide 31 to overcome the bias force of the catch 44 and advance from the locking portion 42 to the guide portion 40 of the locking channel 38. As a result, the guide 31 slides out of the locking channel 38 into the unlocked position.

The introducer locking hub 30 is then disengaged from the sheath locking sleeve 28 and the introducer 6 can be withdrawn from the central lumen of the sheath 8.

In some procedures, a curved approach to the treatment site is desirable. For example, during a transseptal approach for mitral valve replacement/repair. Mitral valve diseases are among the most prevalent valvular heart diseases and necessitate surgical procedures for the repair or replacement of this valve. Conventional left atriotomy is the standard approach for most surgeons. However, the transseptal approach can confer better exposure to the mitral valve in cases where the left atrium is small, where there are adhesions caused by previous procedures, where there are associated operations requiring right atriotomy, and where there is beating heart surgery.

In the transseptal approach, the right atrium is opened and a longitudinal incision (approximately 4 cm) made in the middle of the foramen ovalis on the intra-atrial septum. The septal edges are then pulled in order to expose the mitral valve fully. Conventionally a dilator and/or shunt is required to expand the opening, a shunt can also be used to close the opening and provide an access point for future procedures. However, a major drawback of the transseptal approach surrounds the risk associated with expanding the incision, maintaining the expanded opening the foramen ovalis and the use of a shunt for closing the opening. The sheath of the present disclosure allows for the local and temporary expansion of the incision site only during delivery of the prosthetic device through the incision site.

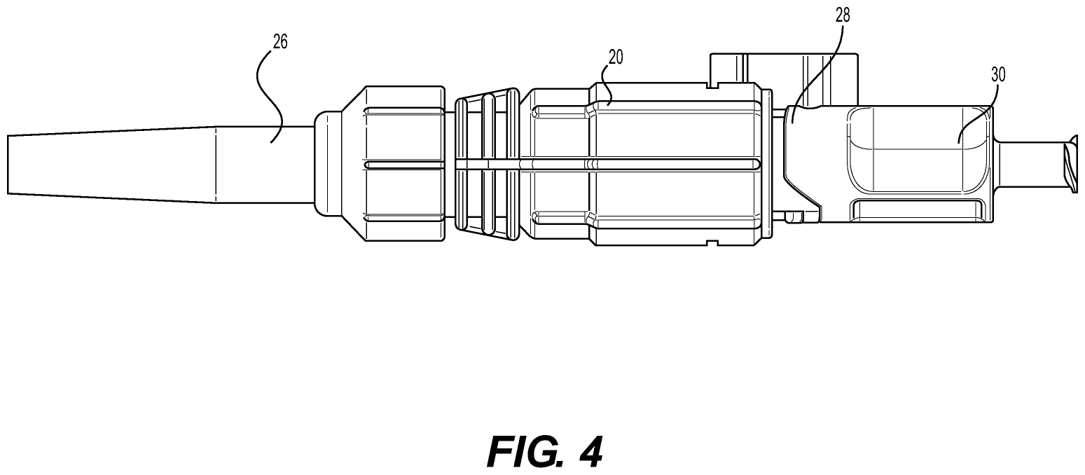

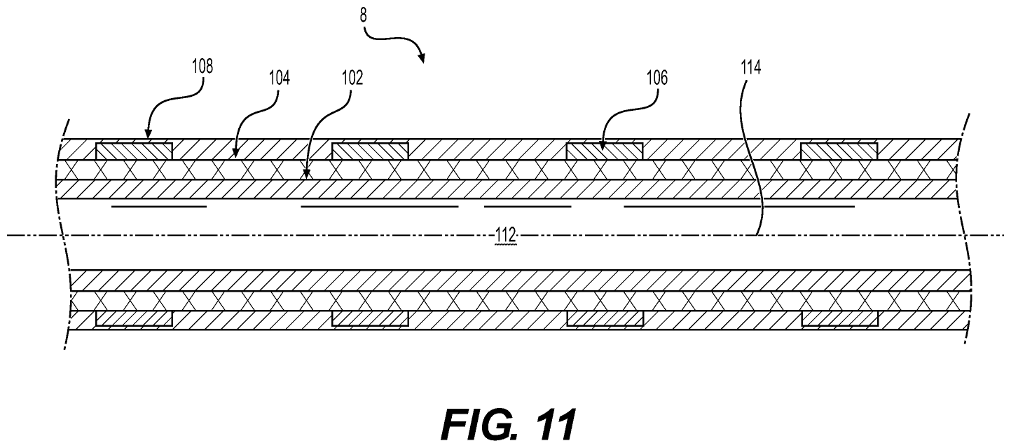

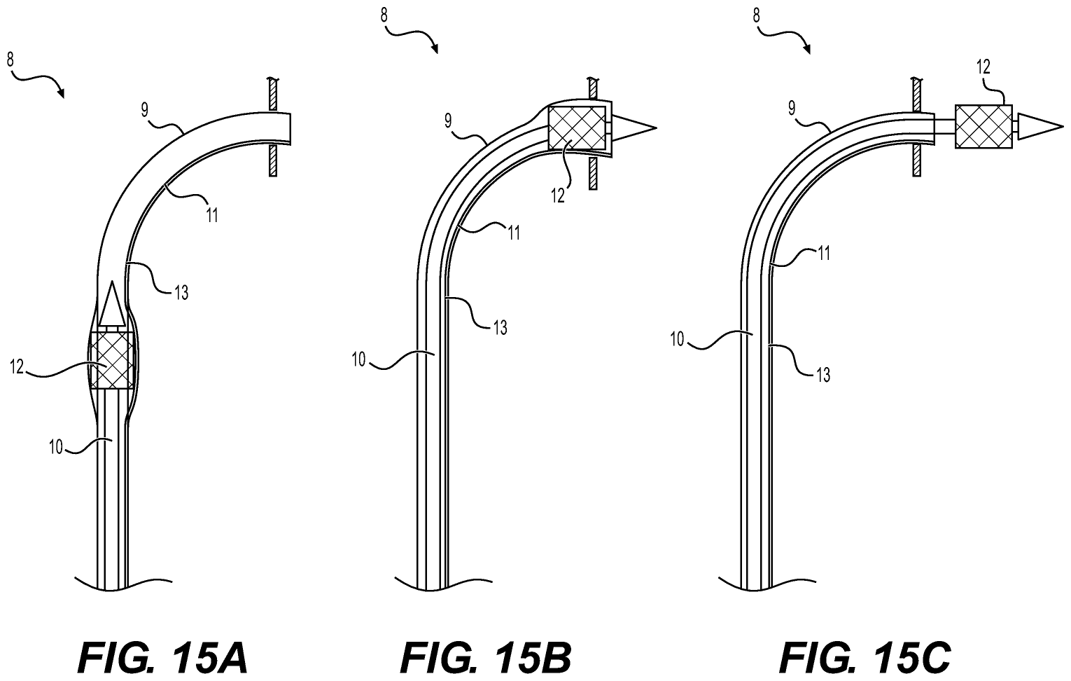

As described above, the sheath assembly 8 and introducer 6 can be used to introduce the delivery apparatus 10 and the prosthetic device (e.g., implant 12) into a patient's body. Disclosed herein is a system and method for bending/curving the distal end of the sheath 8 to allow for a curved approach to the treatment site. As illustrated in FIG. 3, the introducer device/sheath assembly 8 can comprise the sheath hub 20 at a proximal end of the device and an expandable sheath 8 extending distally from the sheath hub 20. The sheath hub 20 can function as a handle for the device. The expandable sheath 8 has a central lumen to guide passage of the delivery apparatus 10 for the medical device/prosthetic heart valve. In an alternative examples, the introducer device/sheath assembly need not include the sheath hub 20. For example, the sheath 8 can be an integral part of a component of the sheath assembly, such as the guide catheter. As described above, the sheath 8 can have a natural, unexpanded outer diameter that will expand locally upon passage of the medical device. In certain examples, the expandable sheath 8 can comprise a plurality of coaxial layers extending along at least a portion of the length of the sheath 8. Example expandable sheaths are described, for example, in U.S. patent application Ser. No. 16/378,417, entitled “Expandable Sheath,” and U.S. Provisional Patent Application No. 62/912,569, entitled “Expandable Sheath,” the disclosures of which are herein incorporated by reference. The structure of the coaxial layers is described in more detail below with respect to FIGS. 11-14. The structure facilitating the curving/bending of the distal end of the sheath 8 is described in reference to FIGS. 15A-15B.

In general, the sheath 8 can include a first tubular layer (inner layer 102) and a resilient elastic tubular second layer 104 radially outward of the inner layer 102, the elastic second layer 104 being configured to apply radial force to the inner layer 102. When a medical device (implant 12) is passed through the sheath 8 (FIG. 15A), the diameter of the sheath 8 temporarily and locally expands from an initial diameter to an expanded diameter around the medical device (implant 12). The sheath 8 resiliently returns to the initial diameter by radial force applied by the elastic second layer 104 upon passage of the medical device (e.g., implant 12). As provided in FIGS. 15A-15B, the distal tip 9 of the sheath 8 is configured to bend is a direction away from a longitudinal axis of the sheath 8. To facilitate bending, the distal tip 9 can be constructed from a more flexible foramen ovalis than the remaining portion of the sheath 8. That is, the distal tip 9 can be constructed from a material having a lower stiffness than a material of the remaining portion of the sheath 8. The distal tip 9 can also include some feature or treatment that would encourage bending. For example, slits or groove could be cut into the outer surface of the sheath 8 along the distal tip 9. The slits/grove would create a weakened portion and voids along the length, encouraging the sheath 8 to curve along this portion. Various methods of bending the distal tip 9 are described below.

The distal tip 9 can be integrally formed with the remaining portion of the sheath 8. That is, the distal tip 9 can be constructed from the same coaxial layered material structure as the remaining portion of the sheath 8. In another example, the distal tip 9 can be coupled to the distal end of the sheath 8. For example, the distal tip 9 can be constructed as a separate working channel, having different material properties than the sheath 8, that is coupled to the distal end of the sheath 8. In these examples, the distal tip 9 can be fixedly coupled to the distal end of the sheath 8. For example, the distal tip 9 can be coupled to the remaining portion of the sheath 8 by a mechanical fastener, a chemical fastener, a thermal process, or suitable means for coupling the distal tip 9 to the sheath 8. Similar to the sheath 8, the distal tip 9 temporarily and locally expands from an initial diameter to an expanded diameter around the medical device (e.g., implant 12). For example, when the medical device is passed through the central lumen of the distal tip 9, the diameter of the distal tip 9 expands from an initial tip diameter to an expanded tip diameter around the medical device (implant 12). After, the distal tip 9 resiliently returns to the initial tip diameter upon passage of the medical device. Similar to the sheath 8, and elastic layer can be provided over the distal tip and a radial force applied by the elastic layer urges the distal tip 9 to return to the initial diameter upon passage of the medical device (implant 12).

In an example system, the sheath 8 can include a pull wire to facilitate bending of the distal tip 9. As illustrated in FIG. 15, the sheath 8 (and distal tip 9) can include a pull wire lumen 11 extending from the distal tip 9 of the sheath and a proximal end of the sheath 8 and a pull wire 13 extending therethrough. The pull wire lumen 11 can be embedded within the wall thickness of the sheath 8. In another example, the pull wire lumen 11 is provided along the central lumen of the sheath 8. In a further example, the pull wire lumen 11 is provided along the outer surface of the sheath 8. It is further contemplated that the pull wire 13 can extend through the central lumen of the sheath 8 or along the outer surface of the sheath 8. In these examples, a guide feature may be provided along the length of the central lumen and/or outer surface of the sheath 8 to ensure the pull wire 13 does not stray from its intended routing along the sheath 8. Regardless of position (withing wall thickness, within central lumen, exterior of sheath 8), the pull wire lumen 11/pull wire 13 is laterally offset from the longitudinal axis of the sheath 8. In general, the pull wire 13 extends along a side of the sheath 8. A force applied to the pull wire causes the distal tip 9 of the sheath 8 to approximate a curved shape. Tension on the pull wire 13 will curve the distal tip 9 of the sheath 8 in a direction corresponding to the pull wire 13. For example, the pull wire 13 can extend along a first side (e.g., right) of the sheath 8. Tension application to the pull wire 13 will cause the sheath 8 (at the distal tip 9) to curve in a direction corresponding to the first site of the sheath (e.g., curve to the right, away from the longitudinal axis of the sheath 8). Similarly, release of the tension on the pull wire 13 will cause the sheath 8 to straighten and return towards the original straight profile.

The pull wire 13 can be coupled to the distal tip 9 at a coupling point proximate the distal end of the sheath. The pull wire can be coupled at the distal end surface of the sheath 8 or at a location proximate the distal end surface. In another example, the coupling point for the pull wire is offset from the distal end of the sheath 8. The pull wire can be coupled to the sheath 8/pull wire lumen 11 but a mechanical fastener (e.g., an anchor, a clip, a pin) and/or chemical fastener. The pull wire can also be coupled to the sheath/pull wire lumen by a heat treatment process.

In another example (not shown), a curved stylet may be used to curve the distal tip 9 of the sheath 8. For example, a curved stylet maybe provided that is movable within the central lumen of the sheath 8. The distal end of the stylet can include a curved portion that, when received within the central lumen of the sheath 8, effects a corresponding curvature of the sheath 8. The stylet can be movable within the sheath 8 to a final position such that the curved portion of the stylet is proximate the distal tip 9 of the sheath 8 and the stylet effects a corresponding curvature of the distal tip 9. In general, the stylet may include a central lumen for receiving/passing over a guidewire or other medical device.

Various features of the coaxial layer structure of the sheath 8 are described in reference to FIGS. 11-14. With reference to FIG. 11, the expandable sheath 8 can include an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114.

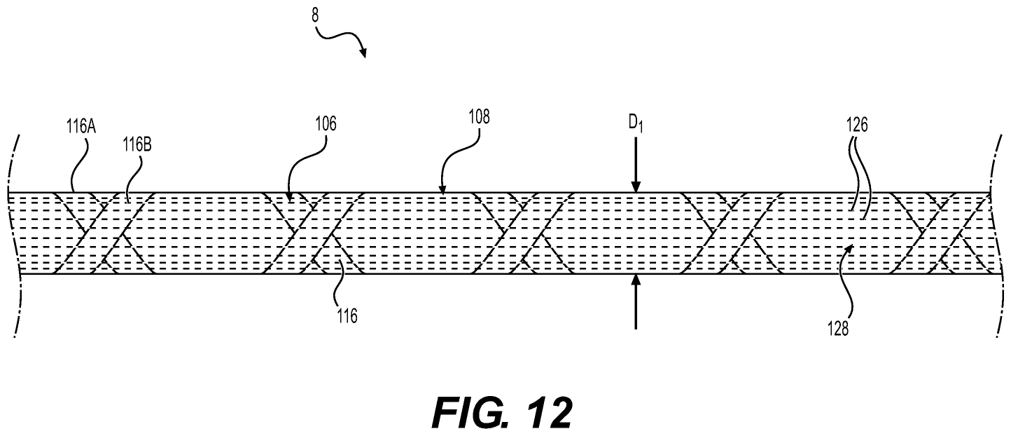

Referring to FIG. 12, when the sheath 8 is in an unexpanded state, the inner layer 102 and/or the outer layer 108 can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D1, the ridges 126 and the valleys 128 can level out or be taken up as the surface radially expands and the circumference increases, as further described below. When the sheath collapses back to its natural diameter, the ridges 126 and valleys 128 can reform.

In certain examples, the inner layer 102 and/or the outer layer 108 can comprise a relatively thin layer of polymeric material. For example, in some examples the thickness of the inner layer 102 can be from 0.01 mm to 0.5 mm, 0.02 mm to 0.4 mm, or 0.03 mm to 0.25 mm. In certain examples, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular examples, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some examples of the sheath 8 can include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some examples of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidine fluoride), are also suitable for use with the sheath 8. Such hydrophilic coatings may also be included on the inner surface of the inner layer 102 to reduce friction between the sheath and the delivery system, thereby facilitating use and improving safety. In some examples, a hydrophobic coating, such as Perylene, may be used on the outer surface of the outer layer 108 or the inner surface of the inner layer 102 in order to reduce friction.

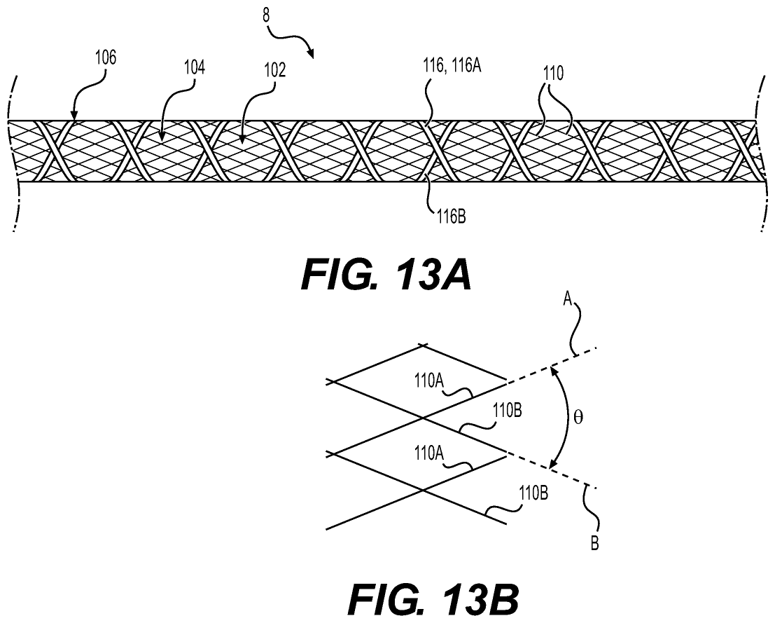

In certain examples, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (e.g., metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain examples, the angle θ can be from 5° to 70°, 10° to 60°, 10° to 50°, or 10° to 45°. In the illustrated aspect, the angle θ is 45°. In other examples, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular examples, the filaments 110 can be wires made from metal (e.g., Nitinol, stainless steel, etc.), or any of various polymers or polymer composite materials, such as carbon fiber. In certain examples, the filaments 110 can be round, and can have a diameter of from 0.01 mm to 0.5 mm, 0.03 mm to 0.4 mm, or 0.05 mm to 0.25 mm. In other examples, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for certain examples. If braided wire is used, the braid density can be varied. Some examples have a braid density of from ten picks per inch to eighty picks per inch, and can include eight wires, sixteen wires, or up to fifty-two wires in various braid patterns. In other examples, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In certain examples, the elastic third layer 106 can be configured to apply force to the underlying inner layer 102 and second layer 104 in a radial direction (e.g., toward the central axis 114 of the sheath) when the sheath expands beyond its natural diameter by passage of the delivery apparatus 10 through the sheath. Stated differently, the elastic third layer 106 can be configured to apply encircling pressure to the layers of the sheath beneath the elastic third layer 106 to counteract expansion of the sheath. The radially inwardly directed force is sufficient to cause the sheath to collapse radially back to its unexpanded state after the delivery apparatus 10 is passed through the sheath.

In the illustrated aspect, the elastic third layer 106 can comprise one or more members configured as strands, ribbons, or elastic bands 116 helically wrapped around the braided second layer 104. For example, in the illustrated aspect the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some examples, the elastic layer can comprise an elastomeric material having a modulus of elasticity of 200 MPa or less. In some examples, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other examples, the elastic third layer 106 can also be radially outward of the polymeric outer layer 108.

In certain examples, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal ridges 126 or folds in the inner layer 102 and outer layer 108, can allow the lumen 112 of the sheath to expand as a prosthetic device is advanced through it.

For example, the inner layer 102 and the outer layer 108 can be heat-bonded during the manufacturing process such that the braided second layer 104 and the elastic third layer 106 are encapsulated between the inner layer 102 and outer layer 108. More specifically, in certain examples the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal and/or distal ends of the sheath. In certain examples, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the sheath.

FIG. 13 illustrates radial expansion of the sheath 8 as a prosthetic device (e.g., implant 12) is passed through the sheath in the direction of arrow 132 (e.g., distally). As the prosthetic device (implant 12) is advanced through the sheath 8, the sheath can resiliently expand to a second diameter D2 that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted above, the inner layer 102 and/or the outer layer 108 can resist axial elongation such that the length L of the sheath remains constant, or substantially constant. This can reduce or prevent the braided layer second 104 from lengthening, and thereby constricting the lumen 112.

Meanwhile, the angle θ between the filaments 110A and 110B can increase as the sheath expands to the second diameter D2 to accommodate the prosthetic valve. This can cause the braided second layer 104 to foreshorten. However, because the filaments 110 are not engaged or adhered to the inner layer 102 or outer layer 108, the shortening of the braided second layer 104 attendant to an increase in the angle θ does not affect the overall length L of the sheath. Moreover, because of the longitudinally-extending ridges 126 or folds formed in the inner layer 102 and outer layer 108, the inner layer 102 and outer layer 108 can expand to the second diameter D2 without rupturing, in spite of being relatively thin and relatively non-elastic. In this manner, the sheath 8 can resiliently expand from its natural diameter D1 to a second diameter D2 that is larger than the diameter D1 as a prosthetic device is advanced through the sheath, without lengthening, and without constricting. Thus, the force required to push the prosthetic implant through the sheath is significantly reduced.

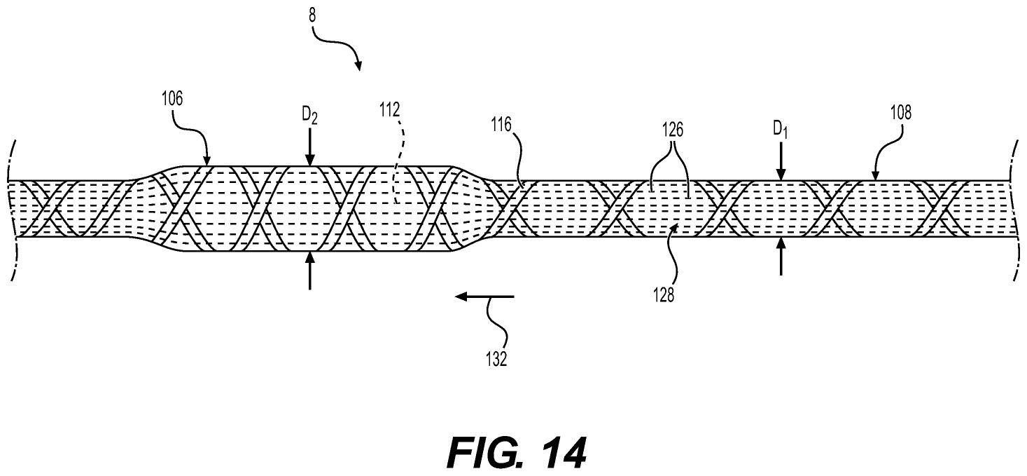

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (e.g., implant 12) can radially collapse back to the initial diameter D1 under the influence of the elastic third layer 106. The inner layer 102 and outer layer 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

A method for controlling articulation/bending of a delivery sheath is disclosed herein. The method includes providing an expandable introducer sheath with a central lumen extending therethrough and a pull wire coupled to the distal end of the sheath. A tension force is applied to a proximal end of the pull wire resulting in a corresponding bending motion/curvature of the sheath in a direction away from the longitudinal axis of the sheath. When the force is released from the pull wire the sheath returns back toward the longitudinal axis of the sheath. In some sheaths the distal tip portion includes a feature (e.g., is constructed from a more flexible material, includes a surface treatment, includes slots/grooves on the outer surface of the sheath) that facilitate curving of the distal tip portion with respect to the remainder of the sheath. In these examples, only the distal tip portion of the sheath curves in response to tension being applied on the pull wire. In another example, the sheath curves along an entire length of the sheath.

Another example method for controlling articulation/bending of a delivery sheath includes providing an expandable introducer sheath with a central lumen extending therethrough, where a distal tip portion of the sheath is more flexible than a proximal portion of the sheath. A stylet is inserted into the central lumen of the sheath, the stylet including a curved portion for effecting a curve on the sheath. When the curved portion of the stylet is aligned with the distal tip portion, the distal tip portion in a direction corresponding to the curvature of the stylet. For example, the stylet may include a curved portion having a curvature extending in a direction away from the longitudinal axis of the sheath. When the curved portion of the stylet aligns with the more flexible portion of the sheath, a corresponding curving effect is accomplished. In some examples, the stylet includes a curved portion that curves in a direction away from the longitudinal axis of the sheath, resulting in a corresponding curvature in the sheath. Removing the stylet, at least partially, from the central lumen of the sheath, such that the curved portion of the stylet is no longer aligned with more flexible portion of the sheath will result in the sheath returning to its original curvature.

A method of delivering a medical device using an articulating introducer sheath is disclosed herein. The method comprises inserting a sheath at least partially into the blood vessel of the patient. In an example method, the sheath is introduced into the patient via the femoral vein. The distal end of the sheath is then advanced to a first location proximate the treatment site. For example, in a transseptal approach for mitral valve repair/replacement, the sheath is advanced via the inferior vena cava into the right atrium. In some examples, a guidewire is positioned at the treatment site and the sheath is advanced over the guidewire.

The distal end of the sheath is curved to facilitate access to the treatment site. In the example of a transseptal approach for mitral valve replacement, it is necessary to curve the end of the sheath to access the mitral valve through the foramen ovalis.

With the end of the sheath curved, the sheath and/or a medical device can be advanced from the first location (within the right atrium) to the treatment site (at the mitral valve). Accessing the treatment site may require creating an opening the heart tissue (e.g., foramen ovalis) of the patient. In this example, a cutting instrument can be advanced through the sheath to create an opening in the patient's heart tissue. Cutting instruments include, for example, a Brockenbrough-type needle. Using the cutting instrument, an incision is made in the heart tissue (e.g., foramen ovalis), the cutting instrument withdrawn, and the distal end of the sheath is advanced through the opening in the patient's heart tissue.

A medical device (e.g., an implant) is advanced through the central lumen of the sheath to the treatment site. Where the distal end of the sheath is provided through an opening in the patient's heart tissue, advancing the medical device to the treatment site includes advancing the medical device, via the sheath, through the opening in the heart tissue.

The sheath is locally expanding from an initial condition/diameter to a locally expanded condition/diameter by the outwardly directed radial force of the medical device against the inner wall of the central lumen of the sheath. The sheath is then locally contracted from the locally expanded condition at least partially back to the initial condition using inwardly directed radial force an elastic feature of the sheath.

With the distal end of the sheath positioned at the treatment site, the medical device is deployed beyond the sheath and delivered to the patient. When the distal end of the sheath provided through an opening in the patient's heart tissue, opening within the heart tissue is expanded by the passing medical device and released back towards its initial condition upon passing of the implant. Where typical transseptal approach procedures require large incision size and the use of a shunt or access tube to maintain the opening in foramen ovalis, the locally expanding, articulating sheath of the present disclosure allows for a much smaller incision opening as only the sheath needs to be inserted (and maintained) in the opening in the foramen ovalis. As a result, the incision size is reduced from approximately 1.5″ to less than 0.5″. Moreover, because the incision is only expanded temporarily during passage of the implant, and a large opening does not have to be maintained (e.g., by shunt), less stress is placed on tissue surrounding the opening. There is also less concern for adverse events post procedure related to closing the incision or leakage around a shunt left in place because the incision is significantly smaller, and shuts are not required.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A sheath locking system comprising: an introducer locking hub comprising a hub body having a proximal end and a distal end and defining a central lumen extending longitudinally between the proximal end and the distal end, and a locking channel disposed on the hub body; and a sheath locking sleeve removably coupled to the introducer locking hub, the sheath locking sleeve comprising a sleeve body having a proximal end and a distal end and defining a central lumen extending longitudinally between the proximal end and the distal end, a guide disposed on an outer surface of the sleeve body, wherein the guide is movable within the locking channel between an unlocked position where the sheath locking sleeve is rotationally and axially movable with respect to the introducer locking hub, and a locked position where the sheath locking sleeve is axially fixed with respect to the introducer locking hub.

Example 2: The system according to any example herein, particularly example 1, wherein the guide protrudes from the outer surface of the sheath locking sleeve and extends at least partially around a circumference of the sheath locking sleeve.

Example 3: The system according to any example herein, particularly examples 1-2, wherein the guide comprises a cylindrical shaped projection extending from the outer surface of the sheath locking sleeve.

Example 4: The system according to any example herein, particularly examples 1-3, wherein a top surface of the guide does not extend beyond the outer surface of the introducer locking hub when the sheath locking sleeve and the introducer locking hub are coupled.

Example 5: The system according to any example herein, particularly example 4, wherein the guide has a height corresponding to a wall thickness of the introducer locking hub proximate the guide when the sheath locking sleeve and the introducer locking hub are coupled.

Example 6: The system according to any example herein, particularly examples 1-3, wherein a top surface of the guide extends beyond the outer surface of the introducer locking hub when the sheath locking sleeve and the introducer locking hub are coupled.

Example 7: The system according to any example herein, particularly example 6, wherein the guide has a height greater than wall thickness of the introducer locking hub proximate the guide when the sheath locking sleeve and the introducer locking hub are coupled.

Example 8: The system according to any example herein, particularly examples 1-7, wherein the central lumen of the sheath locking sleeve is aligned with the central lumen of the introducer locking hub.

Example 9: The system according to any example herein, particularly examples 1-8, wherein the central lumen of the sheath locking sleeve is coaxial with the central lumen of the introducer locking hub.

Example 10: The system according to any example herein, particularly examples 1-9, wherein at least a portion of the sleeve body of the sheath locking sleeve is received within the central lumen of the introducer locking hub.

Example 11: The system according to any example herein, particularly example 10, wherein the portion of the sleeve body includes the guide.

Example 12: The system according to any example herein, particularly examples 10 or 11, wherein the portion of the sleeve body is adjacent the proximal end of the of the sheath locking sleeve, wherein, when the portion of the sleeve body is received within the central lumen of the introducer locking hub, the proximal end surface of the sheath locking sleeve is adjacent a shoulder provided on an inner surface of the central lumen of the introducer locking hub.

Example 13: The system according to any example herein, particularly examples 1-12, wherein the central lumen of the introducer locking hub includes a first portion having a first diameter adjacent the proximal end of the introducer locking hub, and a second portion having a second, larger, diameter adjacent the distal end of the introducer locking hub, wherein at least a portion of the sleeve body of the sheath locking sleeve is received within the second portion of the central lumen of the introducer locking hub.

Example 14: The system according to any example herein, particularly examples 1-13, wherein the locking channel is formed as at least one of a recess or groove in a surface of the introducer locking hub, a slotted opening, or a combination thereof.

Example 15: The system according to any example herein, particularly examples 1-14, wherein the locking channel includes a guide portion and a locking portion, wherein the guide portion is configured to direct the guide in an axial direction along a side wall of the guide portion towards the locking portion upon rotation of at least one of the introducer locking hub and the sheath locking sleeve the wherein the locking portion of the locking channel is configured to securely engage the guide fixing an axial position of the introducer locking hub with respect to the sheath locking sleeve.

Example 16: The system according to any example herein, particularly examples 15, wherein the guide portion of the locking channel extends from the distal end of the introducer locking hub axially towards the proximal end of the introducer locking hub and circumferentially around the introducer locking hub.

Example 17: The system according to any example herein, particularly examples 16, wherein the guide portion of the locking channel extends helically around and along a length of the introducer locking hub.

Example 18: The system according to any example herein, particularly examples 15-17, wherein the locking portion of the locking channel extends at an angle from the end of the guide portion.

Example 19: The system according to any example herein, particularly example 18, wherein the angle between a centerline of the guide portion and a center line of the locking portion is greater than 90-degrees.

Example 20: The system according to any example herein, particularly examples 15-19, wherein the locking portion extends around a portion of the circumference of the introducer locking hub parallel to the distal end of the introducer locking hub.

Example 21: The system according to any example herein, particularly examples 15-20, wherein the length of the guide portion is greater than a length of the locking portion.

Example 22: The system according to any example herein, particularly examples 15-21, wherein the locking portion includes a catch, securing the guide within the locking portion of the locking channel.

Example 23: The system according to any example herein, particularly example 22, wherein the catch extends from a side wall of the locking portion towards the center of the locking channel.

Example 24: The system according to any example herein, particularly example 1-23, wherein the introducer locking hub includes a second locking channel and the sheath locking sleeve includes a second guide, the second guide movable within the second locking channel between an unlocked and locked position.

Example 25: The system according to any example herein, particularly example 24, wherein the introducer locking hub includes a third locking channel and the sheath locking sleeve includes a third guide, the third guide movable within the third locking channel between an unlocked and locked position.

Example 26: The system according to any example herein, particularly examples 1-25, wherein the distal end of the introducer locking hub includes a tapered surface angled toward an opening of the locking channel.

Example 27: The system according to any example herein, particularly examples 1-26, wherein the distal end of the introducer locking hub includes a first tapered surface angled towards a leading edge of an opening of the locking channel and a second tapered surface angled towards the trailing edge of the opening of the locking channel.

Example 28: The system according to any example herein, particularly examples 1-27, wherein the guide is configured to bias the sheath locking sleeve in a proximal axial direction toward the proximal end of the introducer locking hub when the sheath locking sleeve is rotated in a first axial direction such that the guide advances toward the locking portion of the locking channel into the locked position.

Example 29: The system according to any example herein, particularly examples 1-28, wherein the guide is configured to bias the sheath locking sleeve in a distal axial direction toward the distal end of the introducer locking hub when the sheath locking sleeve is rotated in a second axial direction such that the guide advances away from the locking portion of the locking channel, to the unlocked position.

Example 30: The system according to any example herein, particularly examples 1-29, wherein rotation in the second axial direction causes the guide to bias against the catch and overcome the oppositional forces of the catch retaining the guide within the locking portion of the locking channel.

Example 31: The system according to any example herein, particularly examples 1-30, wherein the sheath locking sleeve is securely couplable to a sheath hub, the sheath hub having an elongated body portion with a central lumen extending therethrough and an expandable sheath coupled to a distal end of the body portion, where a central lumen of the expandable sheath is aligned with the central lumens of the sheath hub, the sheath locking sleeve, and the introducer locking hub.

Example 32: The system according to any example herein, particularly examples 1-31, wherein a portion of the sheath locking sleeve overlaps axially with the introducer locking hub when the locking channel engages the guide.

Example 33: The system according to any example herein, particularly examples 1-32, wherein the introducer locking hub body further comprises gripping surfaces.

Example 34: The system according to any example herein, particularly examples 33, wherein the gripping surfaces are recessed surfaces in the locking hub body.

Example 35: The system according to any example herein, particularly examples 33-34, wherein the gripping surfaces are provided on opposite sides of the locking hub body.

Example 36: The system according to any example herein, particularly examples 33-35, wherein the gripping surfaces are provided along at least fifty percent of the length of the locking hub body.

Example 37: The system according to any example herein, particularly examples 33-36, wherein the gripping surfaces define a dog-bone shaped outer surface of the introducer locking hub in cross section.

Example 38: The system according to any example herein, particularly examples 1-37, wherein the sheath locking sleeve is formed from rigid plastic.

Example 39: The system according to any example herein, particularly examples 1-38, wherein the sheath locking sleeve is formed from polycarbonate.

Example 40: The system according to any example herein, particularly examples 1-39, wherein the locking hub is formed from rigid plastic.

Example 41: The system according to any example herein, particularly examples 1-40, wherein the locking hub is formed from polycarbonate.

Example 42: The system according to any example herein, particularly examples 1-41, further including an elongated sheath member coupled to the sheath locking sleeve the sheath member extending beyond the distal end of the hub body, the sheath member having a central lumen extending therethrough, the central lumen of the sheath member aligned with the central lumen of the sheath locking sleeve.

Example 43: The system according to any example herein, particularly example 42, wherein the sheath locking sleeve forms a continuous inner lumen with the lumen of the sheath.

Example 44: The system according to any example herein, particularly examples 42-43, wherein the sheath member is coupled to the sheath locking sleeve via a sheath hub, wherein the sheath is coupled to the sheath hub and the sheath hub is coupled to the sheath locking sleeve.

Example 45: The system according to any example herein, particularly examples 1-44 further including: an elongated introducer member coupled to the introducer locking hub, the introducer member extending beyond the distal end of the hub body and through the central lumen of the sheath locking sleeve, the introducer member having a central lumen extending therethrough, the central lumen of the introducer member aligned with the central lumen of the introducer locking hub.

Example 46: The system according to any example herein, particularly example 45, wherein the introducer member is disposed within the central lumen of the sheath member.

Example 47: The system according to any example herein, particularly examples 45-46, wherein the elongated introducer member is received within a recessed opening provided on an interior surface of the locking hub, the recessed opening axially aligned with the central lumen of the locking hub.

Example 48: The system according to any example herein, particularly examples 45-47, wherein the introducer member is fixedly coupled to the introducer locking hub.

Example 49: The system according to any example herein, particularly examples 45-48, wherein the introducer member is coupled to the recessed opening of the locking hub by at least one of a press fit, an interference fit, a snap fit, a mechanical fastener, a weld and an adhesive.

Example 50: The system according to any example herein, particularly examples 45-49, wherein the central lumen of the introducer member has a diameter corresponding to a diameter of the central lumen of the introducer locking hub.

Example 51: The system according to any example herein, particularly examples 45-50, wherein the central lumen of the introducer member has a diameter less than a diameter of the central lumen of the introducer locking hub.

Example 52: The system according to any example herein, particularly examples 45-51, wherein at least a portion of the central lumen of the introducer locking hub has a decreasing taper between the proximal end and the distal end of the hub body.

Example 53: The system according to any example herein, particularly examples 45-52, wherein at least a portion of the diameter of the central lumen of the sheath is about the diameter of the introducer, such that the sheath and introducer can be used to gently expand the patient's vasculature to a diameter corresponding to an outer diameter of the sheath.

Example 54: A method of delivering a prosthetic device to a procedure site, the method comprising: providing an introducer locking hub having an elongated introducer coupled to a hub body of the locking hub, the introducer locking hub including a locking channel disposed in the hub body; advancing a sheath locking sleeve to a position adjacent a distal end of the introducer locking hub such that a guide projecting from an outer surface of the sheath locking sleeve is received within a locking channel opening on the introducer locking hub, the sheath locking sleeve coupled to an expandable delivery sheath, where advancing the sheath locking sleeve to a position adjacent the distal end of the introducer locking hub includes advancing the introducer axially within the central lumen of the expandable delivery sheath; rotating the introducer locking hub in a first direction with respect to the sheath locking sleeve to move the guide along the locking channel into a locked position; inserting the coupled sheath and introducer at least partially into the vasculature of the patient; rotating the introducer locking hub in a second direction with respect to the sheath locking sleeve to slide the guide along the locking channel into an unlocked position; disengaging the introducer locking hub from the locking sleeve; withdrawing the introducer from the central lumen of the sheath; advancing a medical device through the central lumen of the sheath; and delivering the medical device to a procedure site via the central lumen of the sheath.

Example 55: The method according to any example herein, particularly example 54, wherein movement of the guide along the locking channel into a locked position includes: movement of the guide along a guide portion of the locking channel toward a locking portion of the locking channel, where the guide portion of the locking channel extends from the distal end of the introducer locking hub axially towards the proximal end of the introducer locking hub and circumferentially around the introducer locking hub; wherein further rotation of the introducer locking hub directs the guide into the locking portion of the locking channel, the locking portion configured to securely engage the guide and fix the axial position of the introducer locking hub with respect to the sheath locking sleeve.

Example 56: The method according to any example herein, particularly examples 54-55, wherein the locking portion of the locking channel extends at an angle from the end of the guide portion.

Example 57: The method according to any example herein, particularly examples 54-56, wherein the locking portion includes a catch that secures the guide within the locking portion of the locking channel.

Example 58: The method according to any example herein, particularly example 57, wherein rotation of the introducer locking hub in the first direction causes the guide to overcome the bias force of the catch and advance the guide beyond the catch into the locking portion, where the catch secures the guide within the locking portion thereby fixing the axial location of the sheath with respect to the introducer.

Example 59: The method according to any example herein, particularly examples 54-58, wherein rotating the introducer locking hub in the second direction causes the guide to side along the locking channel, from the locking portion toward the guide portion, wherein further rotation of the introducer locking hub in the second direction directs the guide out of the locking portion of the locking channel and through the guide portion to release the introducer locking hub from the sheath locking sleeve.

Example 60: The method according to any example herein, particularly examples 54-59, wherein rotation of the introducer locking hub in the second direction causes the guide to overcome the bias force of the catch and advance from the locking portion to the guide portion of the locking channel.

Example 61: A method of securing a delivery sheath to an introducer in a device for prosthetic heart valve delivery device, the method comprising: providing an introducer locking hub having an elongated introducer coupled to a hub body of the locking hub, the introducer locking hub including a locking channel disposed in the hub body; advancing a sheath locking sleeve to a position adjacent a distal end of the introducer locking hub such that a guide projecting from an outer surface of the sheath locking sleeve is received within a locking channel opening on the introducer locking hub, the sheath locking sleeve coupled to an expandable delivery sheath, where advancing the sheath locking sleeve to a position adjacent the distal end of the introducer locking hub includes advancing the introducer axially within the central lumen of the expandable delivery sheath; rotating the introducer locking hub in a first direction with respect to the sheath locking sleeve to move the guide along the locking channel into a locked position; rotating the introducer locking hub in a second direction with respect to the sheath locking sleeve to move the guide along the locking channel into an unlocked position.

Example 62: The method according to any example herein, particularly example 61, wherein movement of the guide along the locking channel into a locked position includes: movement of the guide along a guide portion of the locking channel toward a locking portion of the locking channel, where the guide portion of the locking channel extends from the distal end of the introducer locking hub axially towards the proximal end of the introducer locking hub and circumferentially around the introducer locking hub; wherein further rotation of the introducer locking hub directs the guide into the locking portion of the locking channel, the locking portion configured to securely engage the guide and fix the axial position of the introducer locking hub with respect to the sheath locking sleeve.

Example 63: The method according to any example herein, particularly examples 61-62, wherein the guide portion of the locking channel extends from the distal end of the introducer locking hub axially towards the proximal end of the introducer locking hub and circumferentially around the introducer locking hub.

Example 64: The method according to any example herein, particularly examples 61-63, wherein the locking portion of the locking channel extends at an angle from the end of the guide portion.

Example 65: The method according to any example herein, particularly examples 61-64, wherein the locking portion includes a catch that secures the guide within the locking portion of the locking channel.

Example 66: The method according to any example herein, particularly example 65, wherein rotation of the introducer locking hub in the first direction causes the guide to overcome the bias force of the catch and advance the guide beyond the catch into the locking portion, where the catch secures the guide within the locking portion thereby fixing the axial location of the sheath with respect to the introducer.

Example 67: The method according to any example herein, particularly examples 61-66, wherein rotating the introducer locking hub in the second direction causes the guide to side along the locking channel, from the locking portion toward the guide portion, wherein further rotation of the introducer locking hub in the second direction directs the guide out of the locking portion of the locking channel and through the guide portion to release the introducer locking hub from the sheath locking sleeve.

Example 68: The method according to any example herein, particularly examples 61-67, wherein rotation of the introducer locking hub in the second direction causes the guide to overcome the bias force of the catch and advance from the locking portion to the guide portion of the locking channel.

Example 69: An expandable introducer sheath for deploying a medical device, comprising: a first layer including a central lumen extending axially therethrough; a resilient elastic layer radially outward of the first layer, the elastic layer being configured to apply radial force to the first layer; and wherein when a medical device is passed through the sheath, the diameter of the sheath expands from an initial diameter to an expanded diameter around the medical device, wherein the sheath resiliently returns to the initial diameter by radial force applied by the elastic layer upon passage of the medical device, wherein a distal tip of the sheath is configured to bend is a direction away from a longitudinal axis of the sheath.

Example 70: The expandable sheath according to any example herein, particularly example 69, wherein the distal tip of the sheath is more flexible than a remaining portion of the sheath.

Example 71: The expandable sheath according to any example herein, particularly examples 69-70, wherein the distal tip is constructed from a material having a lower stiffness than a material of the remaining portion of the sheath.

Example 72: The expandable sheath according to any example herein, particularly examples 69-71, wherein the distal tip is integrally formed with the remaining portion of the sheath.

Example 73: The expandable sheath according to any example herein, particularly examples 69-71, wherein the distal tip is coupled to the remaining portion of the sheath.

Example 74: The expandable sheath according to any example herein, particularly example 73, wherein the distal tip is fixedly coupled to the remaining portion of the sheath.

Example 75: The expandable sheath according to any example herein, particularly example 73, wherein the distal tip is coupled to the remaining portion of the sheath by a mechanical fastener, a chemical fastener, a thermal process, or a combination thereof.

Example 76: The expandable sheath according to any example herein, particularly example 69, wherein the distal tip is radially expandable, wherein when a medical device is passed through the distal tip, a diameter of the distal tip expands from an initial tip diameter to an expanded tip diameter around the medical device.

Example 77: The expandable sheath according to any example herein, particularly examples 69-76, wherein the distal tip resiliently returns to the initial tip diameter by radial force applied by an elastic tip layer upon passage of the medical device.

Example 78: The expandable sheath according to any example herein, particularly example 77, wherein the elastic tip layer comprises the elastic layer extended over a length of the distal tip.

Example 79: The expandable sheath according to any example herein, particularly examples 69-78, wherein the distal tip includes the first layer and the elastic layer.

Example 80: The expandable sheath according to any example herein, particularly examples 69-79 further including: a pull wire lumen extending from the distal tip of the sheath and a proximal end of the sheath; a pull wire fixedly coupled to the distal tip of the sheath and passing through the pull wire lumen; wherein a force applied to the pull wire causes the distal tip of the sheath to approximate a curved shape.

Example 81: The expandable sheath according to any example herein, particularly example 80, wherein the pull wire lumen is laterally offset from the central lumen of the first layer adjacent a first side of the sheath.

Example 82: The expandable sheath according to any example herein, particularly example 81, wherein the sheath curves in a direction towards the first side of the sheath.

Example 83: The expandable sheath according to any example herein, particularly examples 69-82, further including: a curved stylet movable within the central lumen of the first layer, wherein a distal end of the stylet includes a curved portion that, when received within the central lumen of the first layer, effects a corresponding curvature of the sheath.

Example 84: The expandable sheath according to any example herein, particularly example 83, wherein the stylet is movable within the sheath to a final position such that the curved portion of the stylet is proximate the distal tip of the sheath and the stylet effects a corresponding curvature of the distal tip.

Example 85: The expandable sheath according to any example herein, particularly examples 83-84, wherein the stylet includes a central lumen extending therethrough.

Example 86: The expandable sheath according to any example herein, particularly examples 69-85, wherein when the diameter of the sheath expands from the initial diameter to the expanded diameter while resisting axial elongation of the sheath such that a length of the sheath remains substantially constant.

Example 87: The expandable sheath according to any example herein, particularly examples 69-86, wherein the first layer is a first polymeric layer.

Example 88: The expandable sheath according to any example herein, particularly example 87, further comprising: a braided layer radially outward of the first polymeric layer, the braided layer comprising a plurality of filaments braided together; the resilient elastic layer radially outward of the braided layer; a second polymeric layer radially outward of the elastic layer and bonded to the first polymeric layer such that the braided layer and the elastic layer are encapsulated between the first and second polymeric layers; wherein the resilient elastic layer is radially outward of the braided layer, the elastic layer being configured to apply radial force to the braided layer and the first polymeric layer.

Example 89: The expandable sheath according to any example herein, particularly example 88, wherein when the diameter of the sheath expands from the initial diameter to the expanded diameter, the first and second polymeric layers resist axial elongation of the sheath such that a length of the sheath remains substantially constant.

Example 90: The expandable sheath according to any example herein, particularly examples 88-89, the first and second polymeric layers comprise a plurality of longitudinally-extending folds when the sheath is at the first diameter.

Example 91: The expandable sheath according to any example herein, particularly example 90, wherein the longitudinally extending folds create a plurality of circumferentially spaced ridges and a plurality of circumferentially spaced valleys.

Example 92: The expandable sheath according to any example herein, particularly example 91, wherein, as a medical device is passed through the sheath, the ridges and valleys level out to allow the sheath to radially expand.

Example 93: The expandable sheath according to any example herein, particularly examples 88-92, wherein the braided layer comprises a self-contracting material.

Example 94: The expandable sheath according to any example herein, particularly examples 88-93, wherein the elastic layer comprises one or more elastic bands helically wound over the braided layer.

Example 95: The expandable sheath according to any example herein, particularly examples 88-94, wherein the elastic layer comprises two elastic bands wound with opposite helicity.

Example 96: The expandable sheath according to any example herein, particularly examples 88-95, wherein the filaments of the braided layer are movable between the first and second polymeric layers such that the braided layer is configured to radially expand as a medical device is passed through the sheath while the length of the sheath remains substantially constant.

Example 97: The expandable sheath according to any example herein, particularly example 96, wherein the filaments of the braided layer are not engaged or adhered to the first or second polymeric layers.

Example 98: The expandable sheath according to any example herein, particularly examples 88-97, wherein the filaments of the braided layer are resiliently buckled when the sheath is at the first diameter, and the first and second polymeric layers are attached to each other at a plurality of open spaces between the filaments of the braided layer.

Example 99: The expandable sheath according to any example herein, particularly example 98, wherein the first and second polymeric layers are attached to each other at a plurality of open spaces between the filaments of the braided layer.

Example 100: The expandable sheath according to any example herein, particularly example 69, wherein the first layer includes a thick wall portion integrally connected to a thin wall portion, wherein the thick wall portion has a C-shaped cross section with a first longitudinally extending end and a second longitudinally extending end and wherein the thin wall portion extends between the first and second longitudinally extending ends so as to define an expanded central lumen extending axially through the first layer, the expanded central lumen defined by the expanded diameter.

Example 101: The expandable sheath according to any example herein, particularly example 100, wherein the first layer, in a non-expanded condition, extends through the central lumen of the elastic layer with the first longitudinally extending end of the first layer under the second longitudinally extending end of the inner tubular layer.

Example 102: The expandable sheath according to any example herein, particularly example 101, wherein the first layer, in a locally expanded condition, has the first and second longitudinally extending ends radially expanded apart into a less overlapping condition with the thin wall portion extending therebetween to form the expanded central lumen.

Example 103: A method for controlling articulation/bending of a delivery sheath, the method includes: providing an expandable introducer sheath with a central lumen extending therethrough, where a distal tip portion of the sheath is more flexible than a proximal portion of the sheath; applying a force to a pull wire coupled to a distal end of the sheath to bend the distal tip portion in a direction away from a longitudinal axis of the sheath; and releasing the force on the pull wire to return the distal tip portion to back towards the longitudinal axis of the sheath.

Example 104: A method for controlling articulation/bending of a delivery sheath, the method includes: providing an expandable introducer sheath with a central lumen extending therethrough, where a distal tip portion of the sheath is more flexible than a proximal portion of the sheath; inserting a stylet into the central lumen of the sheath, where the stylet includes a curved portion for effecting a curve on the sheath; aligning the curved portion of the stylet with the distal tip portion to curve the distal tip portion in a direction away from the longitudinal axis of the sheath; removing the stylet at least partially from the central lumen of the sheath such that the curved portion of the stylet is no longer aligned with the distal tip portion of the sheath, to return the distal tip portion back towards the longitudinal axis of the sheath.

Example 105: A method of delivering a medical device, the method comprising: inserting the sheath at least partially into the blood vessel of the patient; advancing a distal end of the sheath to a first location proximate the treatment site; curving the distal end of the sheath; advancing the distal end of the sheath to the treatment site; advancing a medical device through the central lumen of the sheath to the treatment site; locally expanding the sheath from an initial condition/diameter to a locally expanded condition/diameter by the outwardly directed radial force of the medical device; locally contracting the sheath from the locally expanded condition at least partially back to the initial condition using inwardly directed radial force an elastic feature of the sheath; and delivering the medical device to the treatment site.

Example 106: The method according to any example herein, particularly example 105, wherein the sheath is introduced to the patient via the femoral vein.

Example 107: The method according to any example herein, particularly examples 105-106, wherein advancing the distal end of the sheath to the treatment site includes creating an opening in the heart tissue of the patient.

Example 108: The method according to any example herein, particularly example 107, wherein advancing the medical device to the treatment site includes advancing the medical device through the opening in the heart tissue.

Example 109: The method according to any example herein, particularly example 108, wherein the opening within the heart tissue is expanded by the passing medical device within the sheath.

Example 110: The method according to any example herein, particularly examples 105-109 further comprising: advancing a cutting instrument to the treatment site and using the cutting instrument to create the opening in the heart tissue.

Example 111: The method according to any example herein, particularly example 110, wherein the cutting instrument is a Brockenbrough-type needle.

Example 112: The method according to any example herein, particularly examples 107-111, wherein the opening comprises an incision in the foramen ovalis.

Example 113: The method according to any example herein, particularly examples 105-112 further comprising: advancing the distal end of the sheath through the opening patient's heart tissue.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

1. A sheath locking system comprising:
an introducer locking hub comprising a hub body having a proximal end and a distal end and defining a central lumen extending longitudinally between the proximal end and the distal end, and a locking channel disposed on the hub body; and
a sheath locking sleeve removably coupled to the introducer locking hub, the sheath locking sleeve comprising a sleeve body having a proximal end and a distal end and defining a central lumen extending longitudinally between the proximal end and the distal end, a guide disposed on an outer surface of the sleeve body,
wherein the guide is movable within the locking channel between an unlocked position where the sheath locking sleeve is rotationally and axially movable with respect to the introducer locking hub, and a locked position where the sheath locking sleeve is axially fixed with respect to the introducer locking hub.

2. The sheath locking system of claim 1, wherein the guide protrudes from the outer surface of the sheath locking sleeve and extends at least partially around a circumference of the sheath locking sleeve.

3. The sheath locking system of claim 1, wherein the central lumen of the sheath locking sleeve is aligned with the central lumen of the introducer locking hub.

4. The sheath locking system of claim 1, wherein the locking channel includes a guide portion and a locking portion,
wherein the guide portion is configured to direct the guide in an axial direction along a side wall of the guide portion towards the locking portion upon rotation of at least one of the introducer locking hub and the sheath locking sleeve,
wherein the locking portion of the locking channel is configured to securely engage the guide fixing an axial position of the introducer locking hub with respect to the sheath locking sleeve.

5. The sheath locking system of claim 4, wherein the locking portion includes a catch, securing the guide within the locking portion of the locking channel.

6. The sheath locking system of claim 5, wherein the guide is configured to bias the sheath locking sleeve in a distal axial direction toward the distal end of the introducer locking hub when the sheath locking sleeve is rotated in a second axial direction such that the guide advances away from the locking portion of the locking channel, to the unlocked position,
wherein rotation in the second axial direction causes the guide to bias against the catch and overcome oppositional forces of the catch retaining the guide within the locking portion of the locking channel.

7. The sheath locking system of claim 4, wherein the sheath locking sleeve is securely couplable to a sheath hub, the sheath hub having an elongated body portion with a central lumen extending therethrough and an expandable sheath coupled to a distal end of the elongated body portion, where a central lumen of the expandable sheath is aligned with the central lumens of the sheath hub, the sheath locking sleeve, and the introducer locking hub.

8. The sheath locking system of claim 1, wherein a portion of the sheath locking sleeve overlaps axially with the introducer locking hub when the locking channel engages the guide.

9. The sheath locking system of claim 1, further including:
an elongated sheath member coupled to the sheath locking sleeve the elongated sheath member extending beyond the distal end of the hub body, the elongated sheath member having a central lumen extending therethrough, the central lumen of the elongated sheath member aligned with the central lumen of the sheath locking sleeve; and
an elongated introducer member coupled to the introducer locking hub, the introducer member extending beyond the distal end of the hub body and through the central lumen of the sheath locking sleeve, the introducer member having a central lumen extending therethrough, the central lumen of the introducer member aligned with the central lumen of the introducer locking hub,
wherein the sheath locking sleeve forms a continuous inner lumen with the lumen of the sheath,
wherein the introducer member is disposed within the central lumen of the elongated sheath member.

10. A method of securing a delivery sheath to an introducer in a device for prosthetic heart valve delivery device, the method comprising:
providing an introducer locking hub having an elongated introducer coupled to a hub body of the introducer locking hub, the introducer locking hub including a locking channel disposed in the hub body;
advancing a sheath locking sleeve to a position adjacent a distal end of the introducer locking hub such that a guide projecting from an outer surface of the sheath locking sleeve is received within a locking channel opening on the introducer locking hub, the sheath locking sleeve coupled to an expandable delivery sheath, where advancing the sheath locking sleeve to a position adjacent the distal end of the introducer locking hub includes advancing the introducer axially within a central lumen of the expandable delivery sheath;
rotating the introducer locking hub in a first direction with respect to the sheath locking sleeve to move the guide along the locking channel into a locked position; and
rotating the introducer locking hub in a second direction with respect to the sheath locking sleeve to move the guide along the locking channel into an unlocked position.

11. The method of claim 10, wherein movement of the guide along the locking channel into a locked position includes:
movement of the guide along a guide portion of the locking channel toward a locking portion of the locking channel, where the guide portion of the locking channel extends from the distal end of the introducer locking hub axially towards a proximal end of the introducer locking hub and circumferentially around the introducer locking hub; and
wherein further rotation of the introducer locking hub directs the guide into the locking portion of the locking channel, the locking portion configured to securely engage the guide and fix an axial position of the introducer locking hub with respect to the sheath locking sleeve.

12. An expandable introducer sheath for deploying a medical device, comprising:
a first layer including a central lumen extending axially therethrough;
a resilient elastic layer radially outward of the first layer, the resilient elastic layer being configured to apply radial force to the first layer; and
wherein when a medical device is passed through the sheath, a diameter of the sheath expands from an initial diameter to an expanded diameter around the medical device,
wherein the sheath resiliently returns to the initial diameter by radial force applied by the resilient elastic layer upon passage of the medical device,
wherein a distal tip of the sheath is configured to bend is a direction away from a longitudinal axis of the sheath.

13. The expandable introducer sheath of claim 12, wherein the distal tip of the sheath is more flexible than a remaining portion of the sheath.

14. The expandable introducer sheath of claim 12, further including:
a pull wire lumen extending from the distal tip of the sheath and a proximal end of the sheath; and
a pull wire fixedly coupled to the distal tip of the sheath and passing through the pull wire lumen;
wherein a force applied to the pull wire causes the distal tip of the sheath to approximate a curved shape.

15. The expandable introducer sheath of claim 12, further including:
a curved stylet movable within the central lumen of the first layer, wherein a distal end of the curved stylet includes a curved portion that, when received within the central lumen of the first layer, effects a corresponding curvature of the sheath.

16. The expandable introducer sheath of claim 15, wherein the curved stylet is movable within the sheath to a final position such that the curved portion of the curved stylet is proximate the distal tip of the sheath and the curved stylet effects a corresponding curvature of the distal tip.

17. The expandable introducer sheath of claim 16, further comprising:
a braided layer radially outward of the first layer, the braided layer comprising a plurality of filaments braided together;
the resilient elastic layer radially outward of the braided layer; and
a second polymeric layer radially outward of the resilient elastic layer and bonded to the first layer such that the braided layer and the resilient elastic layer are encapsulated between the first layer and the second polymeric layer;
wherein the resilient elastic layer is radially outward of the braided layer, the resilient elastic layer being configured to apply radial force to the braided layer and the first layer.

18. The expandable introducer sheath of claim 17, wherein when a diameter of the sheath expands from the initial diameter to the expanded diameter, the first layer and the second polymeric layer resist axial elongation of the sheath such that a length of the sheath remains substantially constant,
wherein the first and second polymeric layers comprise a plurality of longitudinally-extending folds when the sheath is at the initial diameter.

19. The expandable introducer sheath of claim 17, wherein the filaments of the braided layer are movable between the first layer and second polymeric layer such that the braided layer is configured to radially expand as a medical device is passed through the sheath while a length of the sheath remains substantially constant.

20. The expandable introducer sheath of claim 19, wherein the first layer includes a thick wall portion integrally connected to a thin wall portion, wherein the thick wall portion has a C-shaped cross section with a first longitudinally extending end and a second longitudinally extending end and wherein the thin wall portion extends between the first and second longitudinally extending ends so as to define an expanded central lumen extending axially through the first layer, the expanded central lumen defined by the expanded diameter.
